(19) United States
(12) Patent Application Publication
(10) Pub. No.: US 2024/0148701 A1
(43) Pub. Date: May 9, 2024

(54) WOULD HEALING METHODS

(71) Applicants: The University of Sydney, New South Wales (AU); Northern Sydney Local Health District, New South Wales (AU)

(72) Inventors: Gemma Figtree, New South Wales (AU); Kristen Bubb, New South Wales (AU); Belinda Di Bartolo, New South Wales (AU)

(21) Appl. No.: 18/278,168

(22) PCT Filed: Feb. 22, 2022
(86) PCT No.: PCT/AU2022/050129

§ 371 Date: Aug. 21, 2023

(30) Foreign Application Priority Data
Feb. 22, 2021 (AU) 2021900465

(51) Int. Cl. A61K 31/426; A61K 31/36; A61P 17/02

(52) U.S. Cl. CPC A61K 31/426; A61K 31/36; A61P 17/02

(57) ABSTRACT

The present invention relates to methods for promoting wound healing in a subject, comprising the administration of a β3-Adrenergic Receptor (β3AR) agonist to a subject in need thereof.

### RELATED APPLICATION

This application claims priority from Australian provisional application AU 2021900465 filed 22 Feb. 2021, the entire contents of which are incorporated herein by reference.

### FIELD OF THE INVENTION

The present invention relates to wound repair and healing, particularly to dermal or cutaneous wounds including but not limited to acute and chronic wounds, burns and ulcers.

### BACKGROUND OF THE INVENTION

Impaired wound healing is a growing clinical problem, most evident in the remarkable numbers of chronic wounds in the aging population: 6.5 million have chronic skin ulcers caused by pressure, venous stasis, or diabetes mellitus costing the health care system a staggering $9 billion annually. There is thus a need for treatments that can improve healing of such chronic wounds.

Wound healing is a complex process which involves and interplay between epidermal and dermal cells, intracellular matrix, angiogenesis, plasma proteins, and the local production of cytokines and growth factors. It is generally accepted that a normal physiological response to injury is a wound repair process that is complete with evidence of collagen type I deposition by about 3 to 4 weeks from injury. A protraction of the wound repair process beyond this time increases the likelihood of formation of a chronic wound.

Diabetes affects over 450 million people worldwide and is expected to increase to 700 million by 2045. People that suffer from diabetes also commonly experience a series of cardiovascular co-morbidities. In particular, diabetics suffer from impaired wound healing capacity due at least in part to microvascular dysfunction and disrupted blood flow, especially in the lower legs. As a result, diabetic patients commonly suffer from prolonged presence of ulcers or wounds on their feet, which are prone to further complications of infection and in severe cases patients require amputation.

There remains a need for improved methods and compositions for use in dermal and cutaneous wound repair, particularly in conditions, such as diabetes, where normal physiological wound healing is impaired.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention seeks to address one or more of the above mentioned needs and in one embodiment provides methods for promoting wound healing in a subject, the method comprising the step of administering a therapeutically effective amount of a β3-Adrenergic Receptor (β3AR) agonist to a subject in need thereof, thereby promoting wound healing in the subject.

The promotion of wound healing may be for the prevention, pre-emptive therapy and/or treatment of a dermal or cutaneous wound, or other wound of the mucous membranes or connective tissues of the subject.

The present invention therefore provides a method for the treatment of a dermal or cutaneous wound, the method comprising the step of administering a therapeutically effective amount of a β3AR agonist to a subject in need thereof, thereby treating the dermal or cutaneous wound.

Preferably, the inducing or promoting of wound repair comprises inducing or promoting angiogenesis and blood flow to a wound. Accordingly, in a further embodiment, the present invention provides a method for promoting revascularisation and blood supply to a wound, the method comprising administering to a subject in need thereof, a therapeutically effective amount of a β3AR agonist.

In another embodiment there is provided a method of decreasing the wound area or volume of a dermal or cutaneous wound, the method comprising the step of administering a therapeutically effective amount of a β3-Adrenergic Receptor (β3AR) agonist to a subject in need thereof, thereby decreasing the wound area or volume of the dermal or cutaneous wound.

There is also provided a method of accelerating the rate of wound healing, or decreasing the time to completion of wound healing or wound closure, the method comprising the step of administering a therapeutically effective amount of a β3-Adrenergic Receptor (β3AR) agonist to a subject in need thereof, thereby accelerating the rate of wound healing, or decreasing the time to completion of wound healing or wound closure.

In another embodiment there is provided a method of inducing or promoting or initiating a wound repair mechanism in a dermal or cutaneous wound comprising the step of administering a therapeutically effective amount of a β3-Adrenergic Receptor (β3AR) agonist to a subject in need thereof, thereby inducing or promoting or initiating a wound repair mechanism in a dermal or cutaneous wound. In this embodiment, the wound may be a chronic wound which is devoid of, or which has minimal active wound repair mechanisms.

In any embodiment, the β3AR agonist may be administered orally, intravenously, intraarterially, intradermally, subcutaneously or topically. In preferred embodiments, the β3AR agonist is administered to enable contact of the β3AR agonist with a dermal or cutaneous wound. In embodiments where the β3AR agonist is administered to enable contact with a dermal or cutaneous wound, the administration is preferably via a gel, lotion, cream, impregnated sponge, ointment or spray or via intradermal or subcutaneous injection.

Accordingly, in preferred embodiments, the present invention therefore provides a method for the treatment of a dermal or cutaneous wound, the method comprising the step of contacting a dermal or cutaneous wound with a therapeutically effective amount of a β3AR agonist, thereby treating the dermal or cutaneous wound.

In another embodiment there is provided a method of decreasing the wound area or volume of a dermal or cutaneous wound including the step of contacting a dermal or cutaneous wound with a therapeutically effective amount of a β3AR agonist, thereby decreasing the wound area or volume of the dermal or cutaneous wound.

There is also provided a method of accelerating the rate of wound healing, or decreasing the time to completion of wound healing or wound closure, the method comprising the step of contacting a wound with a therapeutically effective amount of a β3AR agonist, thereby accelerating the rate of wound healing, or decreasing the time to completion of wound healing or wound closure.

It will be appreciated that the methods of the invention find application in the treatment of normal wound or in the treatment of wounds in healthy individuals. The invention however, finds particular application in the treatment of wounds that may be characterised by deficient wound repair mechanisms, such as arise when the individual has an underlying condition or pathology that impedes normal wound healing. m

In another embodiment there is provided a method of inducing or promoting or initiating a wound repair mechanism in a dermal or cutaneous wound comprising the step of contacting a dermal or cutaneous wound with a therapeutically effective amount of a β3AR agonist, thereby inducing or promoting or initiating a wound repair mechanism in a dermal or cutaneous wound. In this embodiment, the wound may be a chronic wound which is devoid of, or which has minimal active wound repair mechanisms.

In certain embodiments, the wound is in a subject who has or is at risk of impaired wound healing. The subject may have, or be considered at risk of a vascular disease or condition, such as: peripheral arterial disease (PAD), scleroderma, atherosclerosis. The subject may have type I or type II diabetes.

In any embodiment, the wound may be an acute wound.

In the above described embodiments, the dermal wound may be chronic or acute wound and may arise from pressure, laceration, burn, incision, maceration, crushing, puncture abrasion or like injury.

In any embodiment, the wound may be associated with a vascular condition characterised by decreased blood circulation (ischemia). The wound may be a venous leg ulcer, a venous foot ulcer, an arterial leg ulcer, an arterial foot ulcer or a decubitus ulcer (also known as a pressure ulcer, bed sore or pressure sore). The wound may be associated with diabetes mellitus. The wound may be a diabetic foot ulcer.

Accordingly, the present invention provides a method for the treatment or management of a diabetic ulcer, the method comprising administering to a subject in need thereof, a therapeutically effective amount of a β3AR agonist. The diabetic ulcer is preferably a diabetic foot ulcer.

Further, there is provided a use of a β3AR agonist in the manufacture of a medicament for:
promoting wound healing;
the treatment of a dermal or cutaneous wound;
inducing or promoting angiogenesis and blood flow to a wound;
decreasing the wound area or volume of a dermal or cutaneous wound;
accelerating the rate of wound healing, or decreasing the time to completion of wound healing or wound closure;
inducing or promoting or initiating a wound repair mechanism in a dermal or cutaneous wound; and/or
treating or managing a diabetic ulcer, preferably a diabetic foot ulcer.

In another embodiment there is provided a β3AR agonist for use in:
promoting wound healing;
the treatment of a dermal or cutaneous wound;
inducing or promoting angiogenesis and blood flow to a wound;
decreasing the wound area or volume of a dermal or cutaneous wound;
accelerating the rate of wound healing, or decreasing the time to completion of wound healing or wound closure;
inducing or promoting or initiating a wound repair mechanism in a dermal or cutaneous wound; and/or
treating or managing a diabetic ulcer, preferably a diabetic foot ulcer.

In another embodiment there is provided a β3AR agonist or formulation containing same including an effective amount of a β3AR agonist for use in a method as described herein.

In any embodiment, the β3AR agonist may formulated for administration orally or for injection intravenously, intraarterially, intradermally, subcutaneously or intramuscularly.

In certain embodiments, the effective amount of a β3AR agonist may be administered topically or formulated for topical administration to the wound and/or region of tissue surrounding the wound, thereby contacting the wound with the β3AR agonist.

In another embodiment there is provided a formulation for use in a method described herein, wherein the formulation includes a β3AR agonist. In this embodiment, the formulation is adapted for topical application to a dermal wound. The formulation may be in the form of a gel, ointment, lotion or spray.

In another embodiment there is provided a device, personal care article or dressing formulated or adapted for treatment or management of a dermal wound including an effective amount of a β3AR agonist for treatment or management of a dermal wound. In this embodiment the a β3AR agonist may be provided in the form of a gauze, mesh, sponge or bandage.

In any embodiment, the β3AR agonist is mirabegron, or a pharmaceutically acceptable salt thereof.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: β3AR stimulation promotes angiogenesis in vitro. (A) cell migration by scratch assay over 24 hours in response to increasing concentrations of CL 316,243 in HUVECs. 4× magnification of 96 well plate; *P<0.05, **P<0.01, ***P<0.001, ****P<0.0001 vs vehicle control by 2-way and B) tubule formation in HUVECs grown on reduced growth factor Cultrex extracellular matrix, in response to increasing concentrations of β3 AR agonist CL 316,243. *P<0.05. **P<0.01. vs vehicle control by 1-way ANOVA with Bonferroni post-hoc analysis, n=6 from 3 experiments. All data shown is mean±SEM. Representative images depict data obtained from vehicle control and CL 316,243-treated (100 ng/ml) cells, with panel A showing closure at baseline (left) and after 15 hours (right).

FIG. 2: β3AR-stimulated angiogenesis is NOS-dependent (A) Tubule formation in HUVECs and the effect of L-NAME (300 μmol/L); n=5 (B) cell migration rate in HUVECs and the effect of L-NAME (300 μmol/L); n=4. (C) Tubule formation in human adult dermal microvascular endothelial cells (MVEC) and (D) patient-derived endothelial colony forming cells (ECFC) with β3AR agonist CL 316,243 (100 ng/ml) and β3AR antagonist, SR 5926thuy30A (1 μmol/L) n=3. (E) Representative images in ECFCs and F) participant characteristics and medical history of ECFC source. Mean±SEM; **P<0.01, ****P<0.0001 vs control; #P<0.05, ##P<0.01, ####P<0.0001 vs. CL 316,243 by 1-way ANOVA with Bonferroni post-hoc analysis or 2-way ANOVA. ACE/ARB, angiotensin converting enzyme inhibitor/angiotensin receptor blocker; BMI, body mass index; CACS, coronary artery calcium score; SPS, soft plaque score.

FIG. 3: β3AR stimulation accelerates reperfusion following hind limb ischemia (A) Representative images showing Laser Doppler flux in hindlimbs from mice immediately post-ligation and after 14 days of recovery, treated with vehicle (saline) or CL 316,243 (1 mg/kg/day) (B) Summary data of hind limb perfusion both pre- and post-ligation and in the contralateral control limb shown as raw flux data. (C) Calculated ratio of the ischemic to non-ischemic limbs following 14 days of hind limb ischemia. (D) eNOS activity by radioimmunoassay in hind limb tissue from mice treated with vehicle or CL 316,243, at 14 days post-surgery. (E) Superoxide generation measured by lucigenin enhanced chemiluminescence (20 μmol/L) corrected for background luminescence. (F) Immunoblot expression of hindlimb Nox 2 and Nox 4 with β-actin control, V=vehicle, CL=CL-316,243-treated. Mean±SEM; *P<0.05 vs vehicle by 1-way or 2-way ANOVA with Bonferroni post-hoc analysis; n=8. +++P<0.001 vs. Pre-ligation limb perfusion; ###P<0.001 vs. post-ligation (timepoint 0) reperfusion.

FIG. 4: Effect of β3AR stimulation in hind limb ischemia, as measured by laser doppler imaging, in type 1 diabetic mice. A. Schematic diagram showing the study protocol; B. Representative image in type 1 diabetic mouse at the end of the study (day 28) and C. representative image of CD31 staining (left). Right, ratio of perfusion in ischemic to non-ischemic limbs in citrate buffer control (n=7-8) and type 1 diabetes (T1 D, n=10) mice treated with vehicle (saline) or CL 316,243 (1 mg/kg/day, s.c., 28 days). Mean±SEM; *P<0.05, **P<0.01 ***P<0.001 vs. vehicle by 2-way ANOVA with Bonferroni post-hoc analysis. (C) CD31 expression in hind limbs post-ischemia in control and diabetic mice treated with vehicle or CL 316,243. Representative images show CD31 stain in brown. Mean±SEM; *P<0.05, **P<0.01 vs. vehicle by 1-way ANOVA with Bonferroni post-hoc analysis; n=8-10.

FIG. 5: Modified redox signaling after hind limb ischemia was normalized by β3AR stimulation A. upper panel, representative blot of Nox4 protein expression in hind limb tissue from diabetic mice. Lower panel, quantification; B. upper panel, representative blot of Nox2 protein expression in hind limb tissue from diabetic mice. Lower panel, quantification; C. Left, representative blot of nitrotyrosine expression in hind limb tissue from diabetic mice; Right, quantification of nitrotyrosine expression; In all groups protein expression is shown relative to citrate vehicle non-ischemic limb. Data presented as mean±SEM. Statistical analysis by 1-way ANOVA with Bonferroni post-hoc analysis. #P<0.05 vs. diabetes vs. citrate; *P<0.05, **P<0.01 CL 316,243 vs. vehicle; n=4.

FIG. 6: Glutathionylation of eNOS (eNOS-GSS) in ischemic hind limb samples from type 1 (T1) diabetic mice. A. Representative images of immunoblots (IB) performed on protein fractions following eNOS immunoprecipitation (IP). On the left the expression of GSH, detected at 680 nm is shown and in the middle, the simultaneous expression of eNOS, detected at 800 nm are shown. The right panel shows the merged image of both detection channels. The negative control, -IgG antibody used during IP, is shown only in the top panel. All samples were extracted and run simultaneously. B. Summary data of eNOS glutathionylation shown as the ratio of glutathionylated eNOS to total eNOS in ischemic hind limb samples. C. eNOS relative to β-actin expression in total ischemic hindlimb lysate from immunoblot. D. phosphorylated eNOS (serine 1177) relative to total eNOS in the ischemic hindlimb. Summary data presented as mean±SEM. Statistical analysis by 1-way ANOVA with Bonferroni post-hoc analysis. ##P<0.01 vs. citrate, *P<0.05 vs. vehicle.

FIG. 7: β3AR stimulation improves glucose tolerance and recovery from post-ischemic injury in type 2 diabetes. (A) Schematic of the type 2 diabetes (T2D) protocol. (B) Glucose tolerance tests in a cohort of citrate-buffer control (n=3-4) and T2D mice treated with vehicle or CL 316,243 (n=4-5). (C) Area under the curve (AUC) analysis of glucose tolerance results. (D) Ischemic to non-ischemic ratio of perfusion in citrate-buffer treated control (n=12) and T2D (n=13) mice measured by laser doppler imaging. Data presented as mean±SEM; *P<0.05, **P<0.01, ***P<0.001 vs. vehicle by 2-way ANOVA.

FIG. 8: Topical application of gel containing Mirabegron. Topical application of Mirabegron formulated into a lipogel. This gel was applied to freshly explanted skin for 5.5 hrs. Skin was then snap frozen in liquid nitrogen and cryosectioned at 40 μm thickness. DESI-Mass spec was then used to scan the tissue at 30 μm resolution. Tissue image (blue) was constructed using mass signal 284 which was presented in high quantity through-out the tissue. Mirabegron, as delivered by the gel formulation, was detected using its expected mass signal 397 and overlayed onto the tissue image. The same slide used for DESI-Mass spec was subsequently stained with H&E, confirming deliver through to the dermis. Left panel: control panel: skin explant. Right panel: skin explant treated with Mirabegron.

FIG. 9 A. HUVECs migration assay with Mirabegron. A scratch migration assay was performed using HUVECs in media containing Mirabegron (0.85 nM-850 nM) to assess its effect on migration over 15 hours. Data from 6- and 15-hours post-scratch shown as mean±SEM. No significant difference was seen between concentrations (β>0.05, n=3). Statistical analysis by two-way ANOVA with Bonferroni multiple comparisons test. B. Diabetic and Non-Diabetic HMVECs migration assay with Mirabegron. A scratch migration assay was performed using diabetic and non-diabetic HMVECs in media containing 85 nM of Mirabegron and control to assess the effect of Mirabegron on migration over 4.5 hours. n=2). The results indicate that Mirabegron improves migration of diabetic HMVECs with no effect on the migration of non-diabetic HMVECs.

FIG. 10: Wound healing in a diabetic mouse model with topical Mirabegron and vehicle. A. Wound closure over time in diabetic male mice with Mirabegron or vehicle lipogel applied topically to wound. Significant improvement was seen in Mirabegron topically treated wounds (p=0.009, 1-way ANOVA with repeated measures). There was a significant improvement in wound healing with Mirabegron at day 2 (n=14, p=0.0238). Data shown as mean±SEM (n=12-14). B. Individual data points of Mirabegron or vehicle lipogel treated mice from day 2 displayed over mean±SEM. Statistical analysis by two-way ANOVA with Bonferroni multiple comparisons test.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety. For purposes of interpreting this specification, terms used in the singular will also include the plural and vice versa.

There are currently no therapeutics that specifically aim to treat diabetic vascular complications such as peripheral arterial disease and foot ulcers. Microvascular dysfunction that occurs in diabetic patients largely contributes to the prolonged presence of these conditions and is an appropriate target for treatment. The inventors have shown that stimulation of the beta 3 adrenergic receptor (B3AR) present on blood vessels is advantageous for several parameters of vascular function in both in vitro cell experiments and in vivo pre-clinical experiments.

Specifically, in human umbilical vein endothelial cells, stimulation of the receptor increases the cells' ability to form tubules on matrigel coated plates, indicating improved angiogenic capacity. Additionally, these cells showed increased ability to cover a denuded area in the presence of the B3AR agonist, a model that is indicative of wound repair. In a pre-clinical diabetic mouse model the inventors have shown that ischaemic hind limb muscle has increased angiogenesis and thus improved blood perfusion and healing capacity when the mice were orally administered B3AR agonist. Furthermore, the excised ischaemic muscle from mice treated with the agonist had reduced markers of oxidative stress.

The present invention is therefore based on the finding by the inventors that administration of β-3 adrenergic receptor agonist (β3AR) promotes angiogenesis and subsequently increases blood flow to injured muscle of both healthy and diabetic mice. The inventors have developed a new method for the treatment of slow healing, chronic wounds, particularly in the case of ischemia, such as that observed in diabetes. The present invention therefore represent a new modality for accelerating and improving the healing of wounds in a variety of clinical settings in which wound healing is impaired.

As used herein β3AR refers to the beta-3 adrenergic receptor (also known as ADRB3 or β3 adrenoreceptor). In any embodiment, the β3AR agonist may be a peptide, protein, small molecule, or nucleic acid which agonises the β3AR. As used herein the term “agonist” refers to a compound, the presence of which results in a biological activity of a receptor that is the same as the biological activity resulting from the presence of a naturally occurring ligand for the receptor.

Examples of β3AR agonists are well known in the art. As such, the present invention contemplates the use of any β3AR in accordance with the methods of the invention. The criteria that are used to define a characteristic β3-AR pharmacological response have been defined in several studies and can be summarized as follows. β3-AR has high affinity and potency for selective agonists such as mirabegron, vibegron, solabegron, and ritobegron; partial agonist activity of β1- and β2-AR antagonists, such as CGP12177A, bucindolol, and pindolol; an atypically low affinity for β-AR antagonists such as propranolol and nadolol; and lastly, poor stereoselectivity for reference agonist and antagonist enantiomers in respect to the values reported for traditional β1- and β2-AR. Structurally, most of the ligands share a similar backbone, with three domains: a left- and right-hand side connected by a linker. The left-hand side is typically an arylethanolamine or aryloxypropanolamine, the linker has various structures including both aromatic and aliphatic moieties, the right-hand side typically contains polar and/or ionizable functionalities.

β3AR agonists fall in two classes depending on the time of their discovery: the first-generation compounds such as BRL37344 and CL316,243, were developed in the 1990s while the second-generation followed or were improved later.

In preferred embodiments, the β3AR agonist is a small molecule. The small molecule may be selected from the group consisting of: Amibegron (SR-58611A, Sanofi); BRL-37344; CL-316,243; L-742,791; L-796,568; LY-368,842, Mirabegron (YM-178), Nebivolo, Ro40-2148, Solabegron (GW-427,353, GSK); Vibegron (MK-4618, Kyorin Pharmaceutical Co., Ltd, and Kissei Pharmaceuticals Co Ltd); Ritobegron (KUC-7483; Kissei Pharmaceuticals Co Ltd).

In particularly preferred embodiments, the β3AR agonist is CL-316,243 or Mirabegron (YM-178), most preferably Mirabegron.

The structure of Mirabegron is provided below:

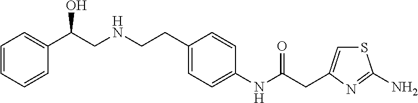 [SMILES: Nc1nc(CC(=O)Nc2ccc(CCNC[C@H](O)c3ccccc3)cc2)cs1]

Mirabegron is also known by the IUPAC name 2-(2-Amino-1,3-thiazol-4-yl)-N-[4-(2-{[(2R)-2-hydroxy-2-phenylethyl]amino}ethyl)phenyl]acetamide, and is registered under CAS number 223673-61-8. Currently, Mirabegron is sold as an oral tablet formulation under the trade names Myrbetriq, Betanis and Betmiga.

Mirabegron can be purchased from Astellas Pharma and was developed for the management of urinary frequency, urinary incontinence or urgency associated with overactive bladder.

### Subjects Requiring Treatment

The methods of the invention can be applied to repair of wounds in essentially any epithelial tissue, including, but not limited to, skin, a genitourinary epithelium, a gastrointestinal epithelium, a pulmonary epithelium, or a corneal epithelium.

Typically, patients who will benefit from the methods of the invention include individuals who may be one at risk for impaired wound repair or impaired wound healing. It will be appreciated that the present invention finds particular application in the treatment of dermal and cutaneous wounds in conditions where ischemia contributes to the slow healing and chronic nature of the wound.

As used herein, the term “impaired wound healing” herein refers to the healing of wounds that do not heal at expected rates including slow-healing wounds, delayed-healing wounds, incompletely healing wounds, dehiscent wounds, and chronic wounds.

As used herein, the phrase “wounds that do not heal at expected rates” refers to wounds that are delayed or difficult to heal. Examples of wounds that do not heal at expected rates include ulcers.

In particular, the individual requiring treatment may be one having systemic or local risk factors for protracted wound repair. Systemic risk factors include systemic infection, metabolic syndrome, diabetes or glucose intolerance, impaired cardiovascular function including peripheral vascular disease, venous stasis disease or other diseases associated with impaired blood flow. Local risk factors include those pertaining to the injury including the nature of the injury itself (for example, a trauma or burn), abnormal inflammation, repeated physical stress by movement, or exposure to UV radiation.

Examples of conditions which increase the risk of impaired wound repair include peripheral arterial disease (PAD) (peripheral vascular disease), obesity and scleroderma. In particularly preferred embodiments, the condition is diabetes, including Type I and Type II diabetes.

The term “peripheral vascular disease” is used interchangeably with “peripheral artery disease”, and herein refers to the obstruction of large arteries not within the coronary, aortic arch vasculature, or brain. PVD can result from atherosclerosis, inflammatory processes leading to stenosis, an embolism, or thrombus formation. It causes either acute or chronic ischemia (lack of blood supply). Often PVD is a term used to refer to atherosclerotic blockages found in the lower extremity.

In the above described embodiments, the dermal wound may be chronic or acute wound and may arise from laceration, burn, incision, maceration, crushing, pressure, puncture abrasion or like injury. The wound may be a chronic skin wound such as a venous stasis ulcer, a diabetic foot ulcer, a neuropathic ulcer, or a decubitus ulcer. In another class of embodiments, the wound results from surgical wound dehiscence. The methods can also be applied to other types of wounds. For example, the wound can comprise a burn, cut, incision, laceration, ulceration, abrasion, or essentially any other wound in an epithelial tissue.

Typically the injury is one arising from insult to dermal, cutaneous or skin tissue. The insult may impact on all layers of dermal tissue, for example on stratum basale (stratum germinativum), stratum spinosum, stratum granulosum, stratum lucidum.

Examples of particular injury include laceration, abrasion, rupture, burn, contusion, compression. The injury may be a burn, including a 1st, 2nd or 3rd degree burn. The injury may be a bedsore or pressure ulcer.

Chronic or “non-healing” wounds, lesions or ulcers arise when a wound generally fails to follow an appropriate timely healing process to achieve the normal sustained and stable anatomic and functional integrity of healed tissue. Generally speaking, a skin lesion which has failed to make at least substantial progress towards healing within a period of at least about three months, or which has become stable in a partially healed state for more than about three months, or a skin lesion which is unhealed after at least about six months is categorized as a chronic or non-healing wound.

In one embodiment of the present invention, the compositions and methods of the present invention are used for the treatment or pre-emptive therapy of lesions showing early signs of developing into non-healing ulcerous skin lesions. Thus, the methods of the invention can be considered methods for preventing the onset of chronic wounds, an in the context of diabetic patients, methods for the prevention of diabetic foot ulcers.

In particularly preferred embodiments, the individual requiring treatment in accordance with the present invention is a diabetic patient that presents with a chronic skin lesion such as a diabetic ulcer or diabetic foot ulcer (DFU).

As used herein, the term “diabetic ulcer” refers to ulcerations, including foot ulcerations, due to vascular complications associated with diabetes. Microvascular disease is one of the complications of diabetes which may lead to ulceration.

The diabetic chronic skin lesions or DFUs are accompanied by other signs and symptoms apart from the failure of the normal healing process. Typical accompanying symptoms of non-healing ulcerous skin lesions include one or more of the features of pain, exudation, malodor, excoriation, spreading of the wound, tissue necrosis, irritation and hyperkeratosis. Such features can be extremely debilitating and embarrassing for the patient, and can seriously harm the patient's quality of life. In severe cases, they may lead to limb amputation or even death. The compositions and methods of the present invention are useful for treating and preventing non-healing ulcers accompanied by these features.

Accordingly in preferred embodiments, the patient requiring treatment according to the methods of the invention may be suffering from Type I diabetes or Type II diabetes, and has a foot ulcer, defined as an open wound anywhere on the foot (heel, mid-foot, and forefoot). As used herein, “treating” a diabetic foot ulcer includes: (a) limiting the progression in size, area, and/or depth of the foot ulcer; (b) reducing size, area, and/or depth of the foot ulcer; (c) increasing rate of healing and/or reducing time to healing; (d) healing of the foot ulcer (100% epithelialization with no drainage); and/or (e) decreased incidence of amputation or slowing in time to amputation.

It will be appreciated, however, that the methods of the invention include the treatment of foot ulcers that are not associated with diabetes. For example, the foot ulcer requiring treatment may be caused by any underlying pathology, including but not limited to neuropathy, trauma, deformity, high plantar pressures, callus formation, edema, and peripheral arterial disease.

In preferred embodiments, the human diabetic foot ulcer is one caused, at least in part, by neuropathy and resulting pressure (weight bearing on the extremity due to lack of feeling in the foot). As is known to those of skill in the art, human diabetic foot ulcers tend to be due to neuropathy and pressure. In a further preferred embodiment, the diabetic foot ulcer comprises one or more calluses.

In a further embodiment, the diabetic foot ulcer is a chronic ulcer. As used herein, a “chronic” foot ulcer is one that has been present for at least 7 days with no reduction in size; preferably at least 14 days; even more preferably, present at least 21 or 28 days with no reduction in size. In a further preferred embodiment that can be combined with any of these embodiments, the chronic foot ulcer has not responded (ie: no reduction in size, area, and/or depth of the foot ulcer; no healing of the foot ulcer) to any other treatment.

In further embodiments, the present invention also provides methods for reducing or delaying the development of diabetic foot ulcer, comprising administering a therapeutically effective amount of a β3AR agonist to a subject in need thereof, wherein the subject has type 2 diabetes and/or one or more risk factors of a vascular disease. Preferably the method reduces or delays severe or moderate diabetic foot ulcer. In this way, the methods of the invention can be said to relate to methods of management of diabetic ulcers.

The invention may include the step of assessing an individual to determine whether the individual or injury site has one or more systemic or local risk factors described above for an impaired wound repair process. Typically, the individual is assessed for one or more systemic or local risk factors applicable to formation of a chronic wound such as those described herein.

Where the individual is assessed as having one or more local or systemic risk factors for an impaired wound repair process, the method may include the further step of selecting the individual for treatment with a β3AR agonist as described herein, to minimise the likelihood of onset of an impaired wound repair process.

Typically, a subject will be considered at risk of formation of a chronic wound, or of impaired wound repair when the subject has or is suspected of having or is at risk of a vascular disease or condition as described herein. The “risk factors of vascular disease” (also referred to herein as “other specified risk factors of vascular disease”) may be selected from the group consisting of microalbuminuria, proteinuria, hypertension, left ventricular hypertrophy, left ventricular systolic dysfunction, left ventricular diastolic dysfunction, and ankle/brachial index <0.9. In some embodiments the “risk factors of vascular disease” may be selected from the group consisting of a) microalbuminuria or proteinuria; b) hypertension and/or left ventricular hypertrophy by ECG or imaging; c) left ventricular systolic or diastolic dysfunction by imaging; and d) ankle/brachial index <0.9. The “risk factors of vascular disease” may be microalbuminuria or proteinuria. The “risk factors of vascular disease” may be hypertension and/or left ventricular hypertrophy by ECG or imaging. The “risk factors of vascular disease” may be left ventricular systolic or diastolic dysfunction by imaging. The “risk factors of vascular disease” may be ankle/brachial index <0.9.

Beneficial response to treatment with a β3AR agonist according to a method described herein, can be assessed according to whether an individual patient experiences a desirable change in disease status. Examples of desirable change in disease status in impaired wound healing include an increase in blood perfusion at the site of the wound, or of the tissue adjacent to the wound; an increase in wound closure; a decrease in inflammatory response; lessening of pain at the wound site.

Administration and Compositions

The methods of the present invention relates to the administration of a therapeutically effective amount of a β3AR agonist.

In any embodiment the β3AR agonist is a pharmaceutically acceptable salt thereof. In certain embodiments, the β3AR agonist is amorphous or the free base.

In any embodiment, a pharmaceutically acceptable salt thereof may include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)), various amino acids, aluminum, calcium, lithium, magnesium, potassium, sodium, zinc, iron, diethanolamine, amines, such as organic amines, N,N′-dibenzylethylenediamine, chloroprocaine, choline, diethanolamine, dicyclohexylamine, ethylenediamine, N-methylglucamine, and procaine.

In any embodiment, the β3AR agonist is mirabegron or a pharmaceutically acceptable salt thereof. In any embodiment mirabegron is an amorphous or the free base thereof. In certain embodiments, the β3AR is mirabegron and the mirabegron is mirabegron hydrochloride.

The phrase “therapeutically effective amount” generally refers to an amount of one or more agonists, or, if a small molecule agonist, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. In the present invention, the result will involve the promotion and/or improvement of wound healing, including rates of wound healing and closure of wounds

The terms “treatment” or “treating” of a subject includes the administration of a β3AR agonist to an individual with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the individual; slowing in the rate of degeneration or decline; making the final point of degeneration less debilitating; or improving a subject's physical or mental well-being.

In any embodiment, the β3AR agonist may be administered orally, intravenously, intraarterially (for example, during vascular surgery/revascularisation procedures) subcutaneously or intramuscularly. The agonist may be administered by intradermal or subcutaneous injection.

Formulations for injection and oral administration are well known in the art. Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

In any embodiment, the β3AR agonist is administered topically. For example, the agonist can be topically administered by application of an ointment, cream, lotion, gel, suspension, spray, or the like comprising the agonist to the wound. As another example, the agonist can be topically administered by application of a dressing comprising the agonist to the wound, e.g., a dressing impregnated with the agonist or having at least one surface coated with the agonist, e.g., a pad or self-adhesive bandage.

As yet another example, the agonist can be topically administered by application of a transdermal device. Either “passive” or “active” transdermal devices can be employed for administration of one or more compositions of the invention, the selection of which will depend in part upon the location for application of the device (e.g., at or proximal to the site of epithelial damage for local administration of, for example, rapidly metabolized compositions, or distal to the site for systemic composition administration). Examples of passive transdermal devices include reservoir-type patches (e.g., in which the composition is provided within a walled reservoir having a permeable surface) and matrix-type patches (in which the composition is dispersed within a polymeric composition).

Active transdermal devices include, but are not limited to, devices employing iontophoresis (e.g., a low voltage electrical current), electroporation (e.g., short electrical pulses of higher voltage), sonophoresis (e.g., low frequency ultrasonic energy), or thermal energy for delivery of the composition. Typically, passive-type transdermal devices would be utilized for application at a current site of epithelial damage, since additional mechanisms for overcoming the epithelial barrier provided by active-type transdermal devices is not necessary. For a review of various transdermal technologies, see Ghosh, Pfister and Yum Eds. (1997) Transdermal and Topical Drug Delivery Systems (CRC Press, London); Potts and Guy (Eds.) (1997) Transdermal Drug Delivery (Marcel Dekker, New York); and Potts and Cleary (1995) Transdermal drug delivery: useful paradigms. J Drug Targ. 3:247-251.

As yet another example, the agonist can be topically administered by introduction of a foam (e.g., a biologically inert or pharmaceutically acceptable foam) or other carrier comprising the agonist to an epithelial-lined cavity comprising the wound.

It will be evident that various means of administration can be combined, for the same or different agonists. Thus, for example, the agonist can be administered both topically and orally or topically and by injection, simultaneously or sequentially, as indicated by the nature and severity of the wound to be treated.

Topical treatment methods, for example, using a paste, gel, cream, oil, lotion, foam, ointment or like substance are particularly useful where the relevant skin region is one that contains a ruptured skin surface, as this permits penetration of the β3AR agonist to the relevant strata of the skin tissue where the fibroblasts reside.

The composition may be provided to the skin generally with a sterile surface, such as a finger or spatula in a layer of no more than about 10 mm thickness, preferably about 3 mm thickness. It may then be rubbed or massaged into the skin region and surrounding area. The application is generally from once per day to once per week, and generally no longer than 20 weeks, or no longer than 12 weeks.

In one embodiment, the β3AR agonist composition may be applied to a solid substrate i.e. a bandage, dressing or the like, and the substrate then fixed to the relevant skin region.

In a further embodiment, the β3AR agonist may be applied to or embedded in a dressing material, such as a hydrogel dressing, enabling penetration of the β3AR agonist to the epidermal layer of the skin. Suitable hydrogel dressings are known to the skilled person. Hydrogel dressings are available as gels, sheets and gels pre-applied to gauze. Purely synthetic hydrogels are frequently made from polyvinyl pyrrolidone, polyacrylamide or polyethylene oxide.

In one embodiment, the hydrogel is MaxGel (comprised of agar and the polymers povidone and polyethylene glycol and having an overall water content of at least 90%). PVA hydrogels may also be used but are less preferred than PVP (povidone)-based hydrogels. MaxGel dressings come in various sizes (between 2.5×6 cm and up to 24×30 cm patches) and is manufactured by Maxford Medical Technical Co. Ltd (Hong Kong). Other brands of hydrogel sheets—Nu-Gel™ (Johnson and Johnson, New Brunswick, NJ), Clear Site (Conmed Corporation, Utica, NY), Aquasorb (DeRoyal, Powell, Tenn), and Hydrogel Patch produced by Tyco-Kendall Healthcare (USA) and Flexderm (Bertek [Dow Hickam] Sugar Land, Tex).

In the context of a diabetic foot ulcer or similar condition, the methods of the invention may comprise administering a topical formulation as often as deemed appropriate, ie: once per day, twice per day, etc. The methods may further comprise administration of the agonist, or salt thereof for as longed as deemed desirable by an attending physician, for example, until healing of the ulcer. For administration, it is preferred that the topical formulation form a continuous film covering the entire area of the ulcer, including the margins. In a preferred embodiment, the topical formulation is applied with a thickness of approximately 0.25 to 2 mm; preferably 0.5 to 1.5 mm; preferably about 1 mm in thickness.

It will be appreciated that the methods of the invention may include more than one mode of administration. For example, a patient requiring treatment may receive simultaneous oral or intra-arterial treatment, in addition to topical treatment.

The methods may further comprise debridement in and around the wound in combination with administration of the peptide and formulations thereof. Debridement of all necrotic, callus, and fibrous tissue is typically carried for treatment of diabetic foot ulcers. Unhealthy tissue is sharply debrided back to bleeding tissue to allow full visualization of the extent of the ulcer and to detect underlying abscesses or sinuses. Any suitable debridement technique can be used, as determined by an attending physician. The wound can then be thoroughly flushed with sterile saline or a non-cytotoxic cleanser following debridement. In another embodiment, the topical formulation comprises about 0.5% to about 4% hydroxyethyl cellulose (HEC) on a weight (mg)/volume (ml) basis, or on a weight/weight (mg) basis. In various further embodiments, the topical formulation may comprise about 1% to about 3% HEC, or about 2% HEC, on a weight (mg)/volume (ml) basis, or on a weight/weight (mg) basis. These formulations comprising low percentage HEC (ie: 2%) matrices provided a 10-fold increase in peptide release over a 24 hour period from formulations such as those comprising 10% carboxymethylcellulose (CMC), a result that 0 would be unexpected to those of skill in the art. Furthermore, the data show that the HEC matrices are more biocompatible than HPMC and CMC formulations tested.

In a preferred embodiment, the β3AR agonist is formulated for topical administration in a formulation that facilitates updake to the dermis and ischemic tissue.

The compositions of the present invention, particularly topical preparations, may include other components, for example preservatives, tonicity agents, cosolvents, complexing agents, buffering agents, antimicrobials, antioxidants and surfactants, as are well known in the art. For example, suitable tonicity enhancing agents include alkali metal halides (preferably sodium or potassium chloride), mannitol, sorbitol and the like. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents include, but are not limited to, glycerin, propylene glycol and polyethylene glycol. Suitable complexing agents include caffeine, polyvinylpyrrolidone, beta-cyclodextrin or hydroxypropyl-beta-cyclodextrin. The buffers can be conventional buffers such as borate, citrate, phosphate, bicarbonate, or Tris-HCl.

The formulation components are present in concentrations that are acceptable to the site of administration. For example, buffers are used to maintain the composition at physiological pH or at slightly lower pH, typically within a pH range of from about 5 to about 8.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Pharmaceutical compositions may be formulated as sustained release formulations such as a capsule that creates a slow release of modulator following administration. Such formulations may generally be prepared using well-known technology and administered by, for example, by subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

The methods for treatment, prevention or alleviation of irritation or lesion such as wounds, ulcers and other lesions of the skin, mucous membranes or connective tissues of the body according to the present invention may include the administration of compositions as defined herein during or after surgery.

In one embodiment of the present invention, the methods for prevention, alleviation and/or treatment of the present invention include a step of treating the tissue in need of treatment with a local anesthetic agent, such as for example lidocaine.

In methods of the present invention, the treatments with a composition as defined herein may be combined with other types of treatment or procedures normally used in the treatment of wounds, ulcers, scars or other lesions, such as for example debridement, surgical wound revision, topical negative pressure treatment (TNPT), frequent change of wound dressing, control of diabetes and/or off-loading in order to reduce edema.

The co-administration of bactericidal or antifungal agents may further facilitate the treatments according to the present invention by preventing or treating infections in wounds, ulcers or other injured sites. In one embodiment of the present invention, the composition s is co-administrated with one or more antibacterial and/or antifungal agents. Such antibacterial and/or antifungal agents may be administered systemically or topically.

### EXAMPLES

### Example 1: Materials and Methods

### Endothelial Cell Culture

Human umbilical vein endothelial cells (HUVECs; Lonza C2519AS, pooled source, Australia) were grown using standard cell culture conditions in endothelial cell growth medium (EGM Plus®, containing 2% fetal bovine serum, Lonza, Australia). All cells were regularly confirmed to be mycoplasma negative. Two different pooled source cell lines were used in experiments and all were used within passages 2-4. Human adult dermal microvascular endothelial cells were also obtained from Lonza (CC-2543, Lonza Australia) and cultured as above but using endothelial growth medium 2-MV bulletkit. Endothelial colony forming cells (ECFCs) were derived from the peripheral blood of participants in the BioHEART study. This registered study (ACTRN12618001322224) complies with the Declaration of Helsinki and the study protocol and design has been approved by the Northern Sydney Local Health District Human Research Ethics Committee (HREC/17/HAWKE/343). Peripheral blood samples were collected from participants following insertion of a venous cannula for a clinically-indicated CT coronary angiogram (CTCA). Coronary artery disease status was obtained from coronary calcification assessment using Gensini scoring as outlined in Kott et al., (2019) BMJ Open, 9: e028649. Blood was transferred into lithium heparin pathology tubes and stored at room temperature. Peripheral blood mononuclear cells (PBMCs) were isolated within four hours of blood collection using a standard gradient-separation Ficoll preparation. Briefly, PBMCs were plated into 0.1% gelatin-coated flasks at a density of 2.5×10⁴ cells/cm² in endothelial cell growth medium containing 2% fetal bovine serum (EGM2 bulletkit, Lonza, Australia). The flasks were cultured in standard conditions for up to 21 days, with regular monitoring for spontaneous growth of ECFCs. Individual cell lines were frozen down in FBS with 10% DMSO and stored in liquid nitrogen. Selected cell lines based on participants coronary artery disease status were thawed for use in tubule formation and the associated health data was extracted from the biobank database.

For assessing the effect of Mirabegron on cell migration: Human umbilical vein endothelial cells (HUVECs; Lonza, C2519AS) were cultured in Endothelial Cell Growth Media containing 2% fetal bovine serum (EGM2) (Lonza, CC-3162) at standard conditions of 37° C. and 5% C02 (SC). Human dermal microvascular diabetic and non-diabetic endothelial cells (db-HMVECs and HMVECs: Lonza, CC2930 and CC2543 respectively) were cultured in Microvascular Endothelial Growth Media containing 2% fetal bovine serum (FBS) (EGM-2MV) (Lonza, CC-3202) at ST. Cells were between passage 4 and 7 when used.

### Tubule Formation and Cell Migration

Once they were ˜70% confluent, cells were re-suspended in diluted EGM Plus® (1:3) and plated on reduced-growth factor extracellular matrix (15 mg/ml, Cultrex, Trevigen, USA) at a density of 1.5×10⁴ cells/cm². Cells were treated with β3AR agonist, CL 316,243 at concentrations ranging from 1-1000 ng/ml. Some experiments were conducted in the presence of non-selective NOS inhibitor, Nω-nitro-L-arginine methyl ester (L-NAME, 300 μmol/L) or selective β3AR antagonist SR 59230A (1 μmol/L). Cells were incubated in an EVOS FL Auto Imaging system for 16 hours and tubule formation recorded every hour. Optimal tube formation occurred at the 8-hour timepoint; tubule number at this point was quantified manually using NIH Image J software. For migration studies, HUVECs were plated in a 96 well plate at a density of 6×10⁵ cells/cm² in EGM plus and left to reach confluence. A scratch was performed using a 10 μl sterile pipette and media was replaced with diluted EGM Plus® (1:3, as above). Cells were treated with CL 316,243 at concentrations ranging from 1-1000 ng/ml and images were taken at 3-hourly intervals over a 48-hour period.

### Murine Hind Limb Ischemia Model

All animal procedures were approved by the Sydney Local Health District Animal Ethics committee (approval number 2016/007) and conform to the National Health and Medical Research Council of Australia's Code of Practice for the Care and Use of Animals for Scientific Purposes.

Male C57BL6/J mice 8-10 weeks of age were obtained from Australian BioResources (Moss Vale, NSW) with 12 hour light/dark cycles and free access to water and mouse chow (Specialty Feeds, Australia). Mice were housed in groups of 2-5 in standard cages within a Physical Containment Level 2 laboratory. For in vivo angiogenesis 16 mice underwent the femoral vascular ligation model. Mice were anaesthetized with 1.5-2% isoflurane vaporized in oxygen and constant body temperature was maintained. All mice received pre-operative and 24-hour post-operative analgesia (carprofen, 5 mg/kg s.c). A small incision (˜15 mm) was made in the hind limb skin directly over the femoral vasculature. A portion of the femoral artery and vein, distal to the origin of the profunda femoris artery and proximal to the saphenous artery, were isolated and two ligations were performed using 6-0 silk sutures. The femoral artery and vein were then excised between the ligation sites 27. An osmotic mini-pump (model 1002 [for 14 day protocols] or model 1004 [for 28 day protocols], Alzet, USA) containing either CL 316,243 (1 mg/kg/day) or vehicle (normal saline) was then implanted via the femoral skin opening and tunneled around and positioned in the dorsal flank. The mice were randomized 1:1 to treatment protocols prior to undergoing surgery. The skin was closed with non-continuous suture (Prolene 6-0, Johnson and Johnson Medical, Australia). Hind limb blood flow was assessed prior to ligation (baseline) and immediately after undergoing hind limb ischemia, with subsequent imaging at day 3, 7, 10, 14, 21 and 28 post hind limb ischemia. Some experiments were stopped at 14 days for assessment of hind limb histology and biochemistry. Software was used to analyze the perfusion flux (Moor, UK) and investigators were blinded to the treatment groups during the period of analysis. The rate of reperfusion in the hind limb was calculated as a ratio of blood flow in the ischemic versus non-ischemic contralateral limb.

Type 1 diabetes model: 20 C57BL6/J mice 6-8 weeks of age were injected with streptozotocin on 5 consecutive days (55 mg/kg, i.p) to induce pancreatic islet destruction with subsequent hyperglycemia as described in Prakoso et al., (2017) Clin. Sci (Lond), 131: 1345-1360. 16 Non-diabetic control mice received vehicle injections (0.1 mol/L sodium citrate buffer, pH 4.5, i.p). Mice were monitored weekly and blood glucose was measured using a handheld glucometer (Roche Accu-chek) with a blood sample obtained via tail prick. Four weeks after the last injection, mice were randomized (1:1) to receive CL 316,243 or vehicle treatment and underwent hind limb ischemia and minipump implantation as described above. Following randomization 1 control mouse allocated to vehicle treatment died during a procedure due to equipment failure and 1 diabetic mouse randomized to the CL 316,243 group did not recover from surgery.

Type 2 diabetes model: 30 C57BL6/J mice at 6 weeks of age were injected with streptozotocin on 3 consecutive days (55 mg/kg i.p) and concurrently transitioned onto a high fat diet 29 (42% energy intake from lipids, SF04-001, Specialty Feeds, Australia). 24 Non-diabetic time-matched controls were injected with citrate buffer vehicle and fed standard rodent chow. Mice were kept for 20 weeks on high fat diet prior to undergoing hind limb ischemia as described above. Mice were randomized 1:1 to receive CL 316,243 or saline vehicle and this was implanted during hind limb ischemia surgery as outlined above. Following randomization 1 diabetic mouse, allocated to CL 316,243, died during surgery and 1 control mouse, allocated to CL 316,243, did not recover from surgery. 4 diabetic mice (2 per group) were excluded from the study due to blood glucose levels dropping below 15 nmol/L.

Glucose tolerance testing was conducted in fasted type 2 diabetic mice. Rodent chow was removed overnight and testing was conducted in the morning. After baseline glucose testing mice were injected intraperitoneal with sterile D-glucose (2 g/kg). Repeated blood glucose sampling was conducted every 15-30 minutes for 2 hours.

### Type 2 Diabetes Model for Assessment of Topical Mirabegron on Wound Closure

Diabetes Induction: All animal procedures were approved by the Sydney Local Health District Animal Ethics committee (Ethics Approval Number: RESP21007) and conform to the National Health and Medical Research Council of Australia's Code of Practice for the Care and Use of Animals for Scientific Purposes.

8-week-old C57BL6 mice were purchased from Animal Resources Centre (Murdoch, WA) and acclimatised to the Kearns Facility over a 1-week period. Mice were group housed (5/cage) within a Physical Containment Level 2 laboratory and were allowed food and water ad libitum. After acclimatisation, 60 mice were weighed daily and given five consecutive daily intraperitoneal injections of Streptozotocin (STZ) solution (55 mg/kg/day) dissolved in sodium citrate (0.1 M) using a standard insulin needle (30 gauge). After the final injection, mice were weighed and blood glucose level (BGL) measured weekly by pricking the most distal point on the tail with a needle to obtain a blood drop, used with an AccuCheck BGL monitor. Mice became become diabetic over 4 weeks post-STZ, as confirmed with a consistent BGL of 15 mmol/L and if greater than 10% weight loss was observed, insulin was delivered via intraperitoneal injection (1 IU in saline). 4 mice were excluded due to severe weight loss during diabetes induction that exceeded ethical protocols.

Wound creation: The mouse wound healing model was adapted from (Dunn et al., 2013), where a splint was used to prevent healing of wounds via contraction and thus more accurately represent the stages of wound healing seen in humans.

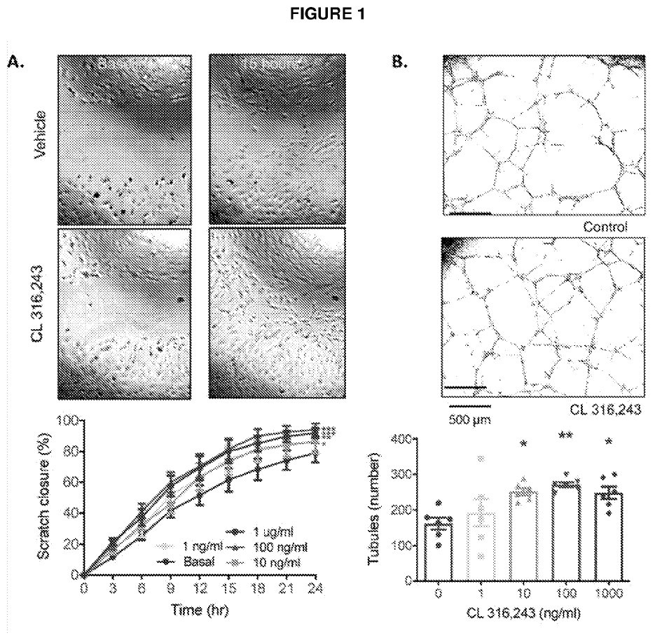

Under anesthesia (2% isoflurane, 98% oxygen), diabetic male mice had hair from upper back removed using clippers to expose skin. Two superficial wounds were created on either side of the midline below shoulders using a 6 mm biopsy punch (Livingstone, LivBioPunch06). Iris scissors were used to create a full thickness circular excision of skin layers including panniculus carnosus. 1 cm diameter circles were cut from 0.5 mm silicone sheets (Thermofisher, P18178) and the biopsy punch was used create a hole in the centre, resulting in toroid shaped splints. These splints were glued and sutured to the skin around the two wounds using superglue (United office choice, 523696) and 6-0 nylon sutures respectively. Wounds and splints were covered with a thin layer of opsite film (Pharmacy Direct, 1005584) to prevent infection and damage from other mice. Following surgery, mice were housed individually for 2 days then group housed (5/cage) for the remainder of the procedure. The methods are illustrated in FIG. 1.

Treatment and measurement: 4 weeks after the final STZ injection and prior to wound surgery, mice were randomised 1:1 to either the topical or systemic group. Mice in each group were further randomised 1:1 to receive either Mirabegron or vehicle control, either delivered topically by applying lipogel or systemically via an osmotic mini pump (Alzet, model 1,002) respectively. For systemic mice, mini pump was then implanted via a single incision in the dorsal flank, tunnelled around to create a pocket for stable positioning, then sutured close. Treatment pumps contained Mirabegron (10 mg/mL) dissolved in DMSO (50%, vol %) and 100% Ethanol (50%, vol %) and put into mini pump, which infused 0.25 μL/h of solution approximating 2 mg/kg/day of Mirabegron per mouse. Vehicle pumps contained DMSO and ethanol only. For mice in the topical group, lipogel was applied daily by removing opsite wound covering and applying the 10 mg/g Mirabegron in lipogel approximating to 2 mg/kg/day of Mirabegron per mouse. Mirabegron lipogel was applied to the right wound and vehicle lipogel to left on the same mouse to be used an internal control. Wounds were measured daily for 12 days by removing opsite covering and using digital callipers to measure wound diameter across 3 axes which were averaged and used to calculate circular wound area. Repeat treatment and vehicle for topical group were applied following daily measurement. Wound measurements were not taken for wounds if splint was no longer attached to prevent invalidation of results due to contractive healing. As such, wound data from 2 mice in topical group were not recorded following day 8 due to splint detachment. Similarly, data from 2 mice in systemic group was excluded from day 10 onwards.

### Histological Analysis

Formalin fixed paraffin-embedded gastrocnemius was cut into 4 μm sections and then sections were deparaffinized. Heat retrieval was performed with Tris-EDTA buffer at pH 9. Slides were incubated overnight with a rabbit polyclonal CD31 antibody (dilution 1:200, Abcam Ltd, Australia) followed by horseradish peroxidase anti-rabbit Envision system (Dako Cytochemistry, Tokyo, Japan). Staining was developed with 3.3 diaminobenzidine tetrahydrochloride (Dako Cytochemistry, Tokyo, Japan) and counterstained with Mayer's hematoxylin stain. Rabbit IgG negative controls (Dako Cytochemistry, Tokyo, Japan) were used. A total of ten non-overlapping images for each gastrocnemius were taken with a light microscope (Leica, DM750 linked to an ICC50 E camera module). Images were taken at ×40 and analyzed with National Institute of Health Image J 1.51j8 software.

### Biochemical Analysis

Hind limb tissue including the gastrocnemius and adductor muscles were isolated and collected at 14 or 28 days. Tissues were separated and implanted in OCT or placed in cryovials and snap-frozen in liquid nitrogen or were fixed in 10% formalin for 24-hours and then moved to 70% ethanol for storage.

### Superoxide Anion Generation

Frozen adductor tissue was prepared for lucigenin-enhanced chemiluminescence assay by homogenising in lysis buffer (250 mM sucrose in phosphate-buffered saline (mM: 129 NaCl, 7 Na2HPO4, 3 NaH2PO4·2H2O, pH 7.4, with protease inhibitors (cOmplete™ EDTA-free, Roche Diagnostics). Sample was added to opaque 96-well plates in the presence of lucigenin (20 μmol/1 N,N′-Dimethyl-9,9′-biacridinium dinitrate) and NADPH (100 μM; β-Nicotinamide adenine dinucleotide 2′-phosphate reduced tetrasodium salt hydrate). The reaction was conducted at room temperature and tracked using a luminometer (Veritas, Turner Biosystems, USA) with an average measurement taken from 20 cycles, as described in Bubb et al., (2018, Microcirulation, e12501). Replicates of each sample were treated with manganese TMPyP (Merck Millipore, Australia, a cell-permeable superoxide dismutase mimetic, 30 μmol/L), during the assay and any signal was subtracted from the total signal as non-superoxide background signal. Superoxide production was normalized to protein concentration or cell count.

### NOS Activity

The activity of NOS was measured using radioimmunoassay according to manufacturer's instructions (Cayman Chemical, USA). Samples were prepared in triplicate and detected using a liquid scintillation counter (5 min detection, Tri-Carb 4910TR 100V, Perkin Elmer, USA). All samples were also assayed in the presence of L-NAME and this was subtracted from the baseline to give a readout of NOS activity.

### Immunoblotting

Gastrocnemius samples were stored at −80° C. and then mechanically homogenized in ice-cold lysis buffer containing 150 mmol/L NaCl, 200 mmol/L Tris-HCl (pH 8.0), 1% Triton X-100, 0.5% deoxycholic acid, 0.1% SDS, N-ethylmaleimide (25 mM) and protease inhibitors (cOmplete™ EDTA-free, Roche Diagnostics). 30 μg of protein lysate was denatured and run under reducing conditions on SDS-PAGE (Bolt™ pre-cast gels and reagents, Thermofisher Scientific, Australia) and transferred onto Immobilon polyvinylidene fluoride membrane (Merck Millipore, Australia). Membranes were incubated in primary antibodies directed at determining protein expression of the following: Nox isoforms (anti-Nox 2, 1:5000; Abcam, Australia; anti-Nox-4, 1:5000; Abcam, Australia); reactive nitrogen species (anti-nitrotyrosine, 1:1000; Abcam, Australia); and both expression and phosphorylation of eNOS (anti Phospho eNOS serine 1177, 1:1000, Cell Signaling Technology, USA; anti-eNOS 1:1000, BD Biosciences, USA) and Akt (anti Phospho Akt 1:1000, Akt 1:1000, Cell Signaling Technology, USA). Specific secondary antibodies recognizing rabbit or mouse primary antibodies were used (IRDye®, Licor; 1:20,000, USA). Membranes were detected using an Odyssey imaging platform (Licor, USA).

### Immunoprecipitation

Gastronemius protein (500 μg) extracted as above was used for co-immunoprecipitation with eNOS. Protein G dynabeads (1.5 mg/ml, 2.8 μm beads, Thermofisher Scientific, Australia) were covalently conjugated with mouse anti-eNOS antibody (BD Biosciences, 1 μg) using bis(sulfosuccinimidyl) suberate amine-amine cross-linking solution (5 mM; ThermoFisher Scientific, Australia). Beads were washed with PBS and incubated with protein lysate overnight at 4° C. IgG controls were prepared using anti-IgG antibodies conjugated to dynabeads using an identical process. Protein was eluted from beads using LDS buffer, denatured and run in non-reduced conditions on 8% Bis-Tris gels using SDS-PAGE and transferred onto polyvinylidene fluoride membrane as above. Expression of oxidized glutathione was detected using mouse anti-glutathione antibody (Virogen, 1:1000). eNOS was detected using rabbit anti-eNOS (Cell Signaling Technology, 1:1000, Australia). Odyssey detection system was used to visualize bands as above.

### Sample Size and Statistical Analysis

Data are expressed as mean±standard error of the mean (SEM). Student's t-test was used for comparison between two groups. For multiple comparisons, 1- or 2-way analysis of variance (ANOVA) was used with Bonferroni post-hoc analysis for multiple comparisons. A P value <0.05 was considered statistically significant. For all mouse studies, sample sizes were calculated based on the 80% power to detect a 30% change in primary endpoint (perfusion ratio) with standard deviation of 25%. Additional mice were added to diabetes groups based on variability and failure rates of diabetes models.

### Example 2: β3AR Stimulation Promotes Angiogenesis

The inventors first established a role for β3AR stimulation in promoting angiogenesis in vitro using HUVECs. The β3AR agonist, CL 316,243, significantly increased migration of HUVECs into the denuded zone (FIG. 1A), with >90% closure reached by 24 hours at the higher concentrations. CL 316,243 also increased the number of tubules formed. This was significantly increased by the 10 and 100 ng/ml concentrations compared to the control (FIG. 1B).

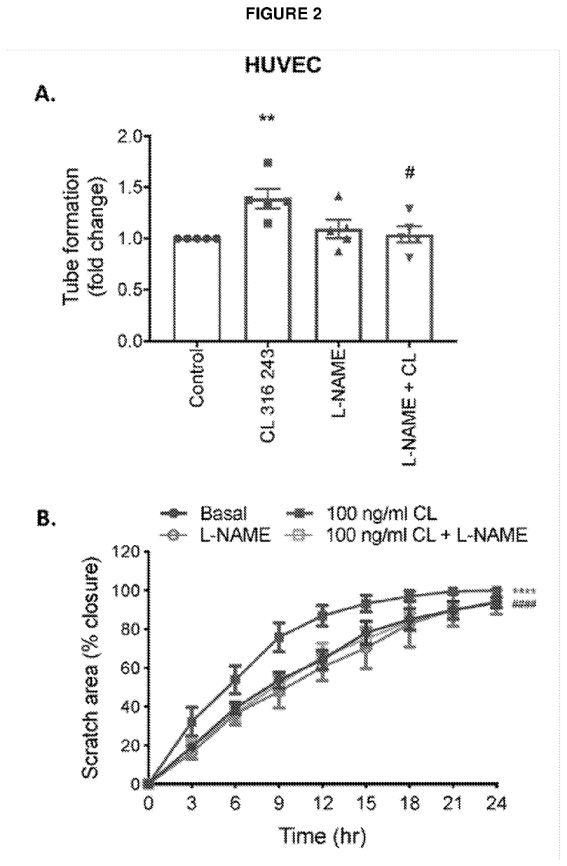

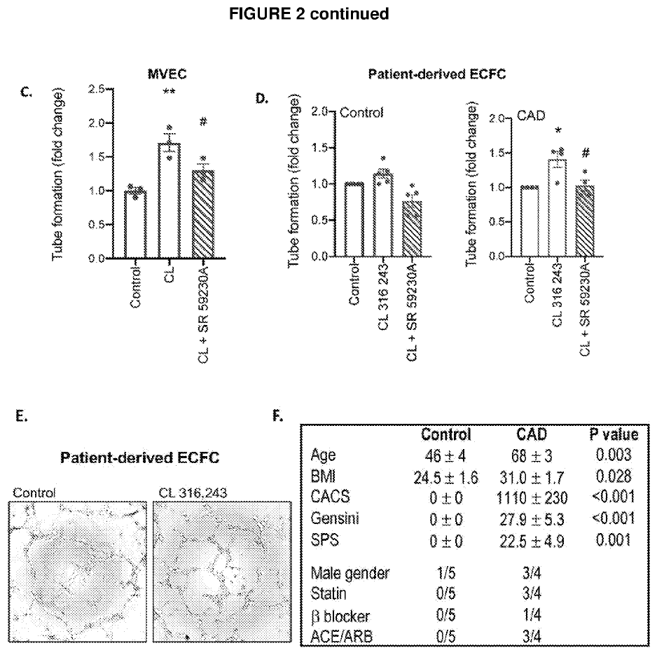

To confirm that β3AR-inducible angiogenesis involves eNOS activation and NO in our system, the inventors exposed HUVECs to L-NAME. The pro-angiogenic effects of β3AR were abolished with L-NAME (FIG. 2A-B). CL 316,243 also stimulated angiogenesis in endothelial cells from adults (dermal source, FIG. 2C), and this effect was b3AR specific, since CL 316,243-induced tubule formation was abolished in response the β3AR antagonist, SR 592230A (FIG. 2C). Furthermore, CL 316,243 could also effectively stimulate angiogenesis via β3 activation in ECFCs derived from patients with significant coronary artery disease (FIG. 2D-F).

### Example 3: 133AR Stimulation Accelerates Reperfusion Following Hind Limb Ischemia

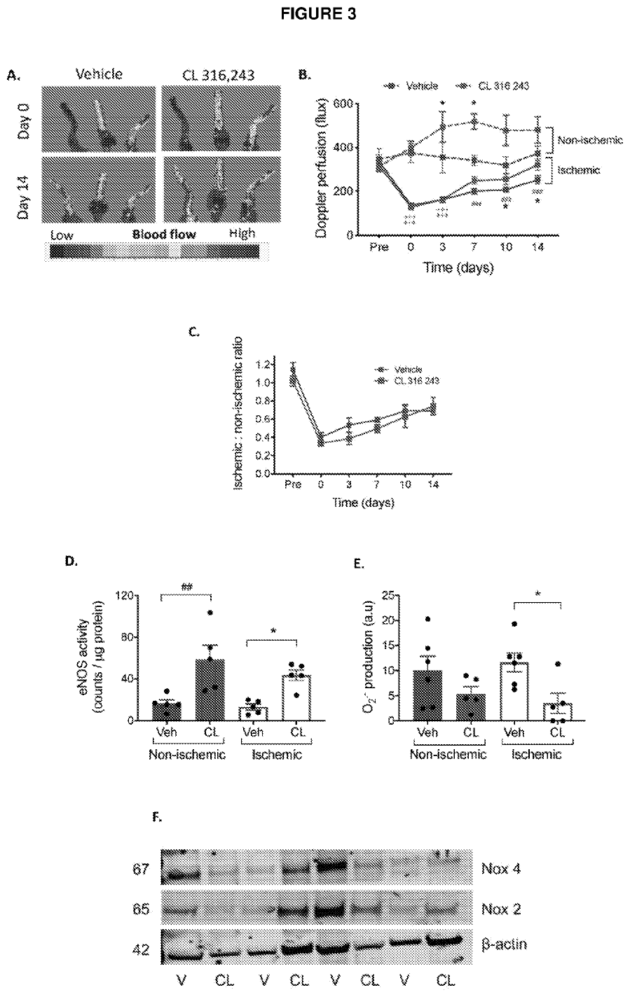

The inventors next examined the angiogenic potential of β3AR in vivo in a model of hind limb ischemia. Ligation of hind limb vascular beds resulted in severely impaired perfusion compared to pre-ligation in both groups (FIG. 3A-B). Although subcutaneous infusion of CL 316,243, but not vehicle, significantly increased perfusion in the ischemic limbs 10-14 days following ischemic injury, systemic infusion of CL 316,243 also increased perfusion in the non-ischemic limb (FIG. 3A-B). When the ischemic non-ischemic ratio was calculated (Krishna et al., 2020, Sci. Rep, 10: 3449), no differences were observed between vehicle and CL 316,243-treated mice (FIG. 3C). The inventors next determined whether CL 316,243 infusion increased eNOS activity in both control and ischemic limbs, thus contributing to the increased perfusion. Indeed CL 316,243 enhanced eNOS activity in both the ischemic and non-ischemic limbs (FIG. 3D). Furthermore, superoxide bioavailability in the ischemic limb was significantly lower after 14 days of CL 316,243 infusion (FIG. 3E) and not related to expression of NADPH oxidase (Nox) isoforms; no significant differences in hind limb protein expression for Nox 2 or 4 were observed (FIG. 3F, fold change from vehicle in ischemic limb: Nox 2, 1.04±0.46; Nox 4 0.88±0.28, n=4, P>0.05).

Collectively, these findings suggest that β3AR stimulation can improve post-ischemic reperfusion by altering NO/redox balance, and also influence perfusion in non-ischemic conditions.

### Example 4: 133AR Stimulation is Effective in Improving Hind Limb Ischemia of Type 1 Diabetic Mice

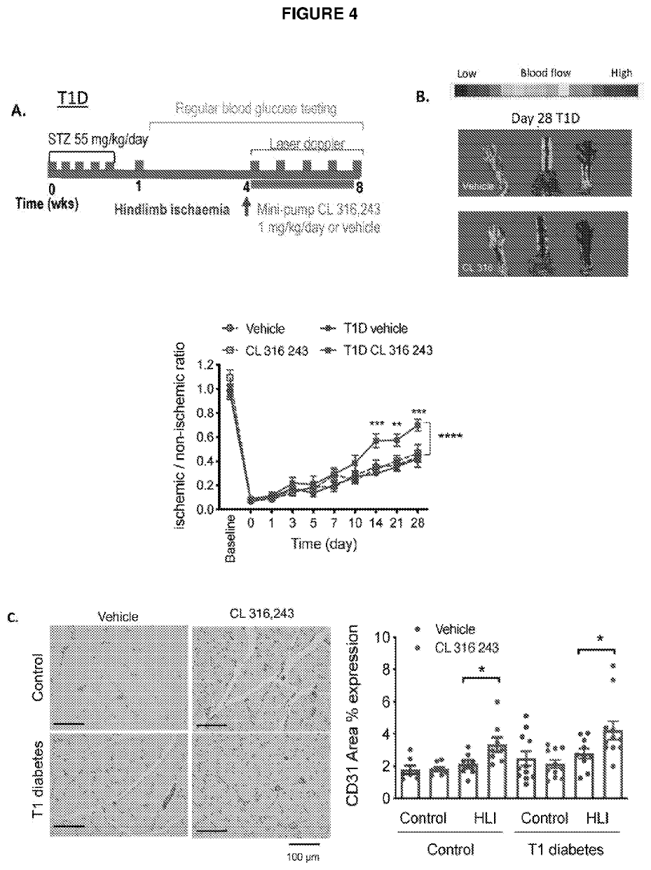

Diabetics have impaired angiogenesis and other vascular complications and are at increased risk of developing PAD. The inventors next examined whether β3AR stimulation could promote angiogenesis in diabetes. The inventors first used a well-validated model of streptozotocin (STZ)-induced type 1 diabetes. Blood glucose levels were significantly elevated within a week of STZ injection in type 1 diabetes mice and remained high for the duration of the 8-week protocol. Hind limb ligation was conducted four weeks after the onset of type 1 diabetes, when the disease phenotype was well-established (FIG. 4A). Type 1 diabetes mice had lower body weight than their non-diabetic counterparts (data not shown). Treatment with the β3AR agonist CL 316,243 had no effect on body weight or non-fasted blood glucose levels (data not shown).

Following hindlimb ischemia, β3AR stimulation resulted in accelerated reperfusion in type 1 diabetes mice, as shown by ˜20 greater ischemic-non-ischemic ratio from 14 days onwards (FIG. 4B). The citrate-buffer treated mice mirrored the results of non-diabetic mice, where perfusion ratio of CL 316,243-treated mice was not different from vehicle controls, and this was also the case from 14-28 days post-ischemia (FIG. 4B). We next assessed vascularization and showed greater CD31+ staining in ischemic hindlimbs of mice treated with CL 316,243, in both the type 1 diabetes and the non-diabetic mice (FIG. 4D).

### Example 5: β3AR Stimulation Ameliorates Dysrequlated Redox Signaling after Hind Limb Ischemia

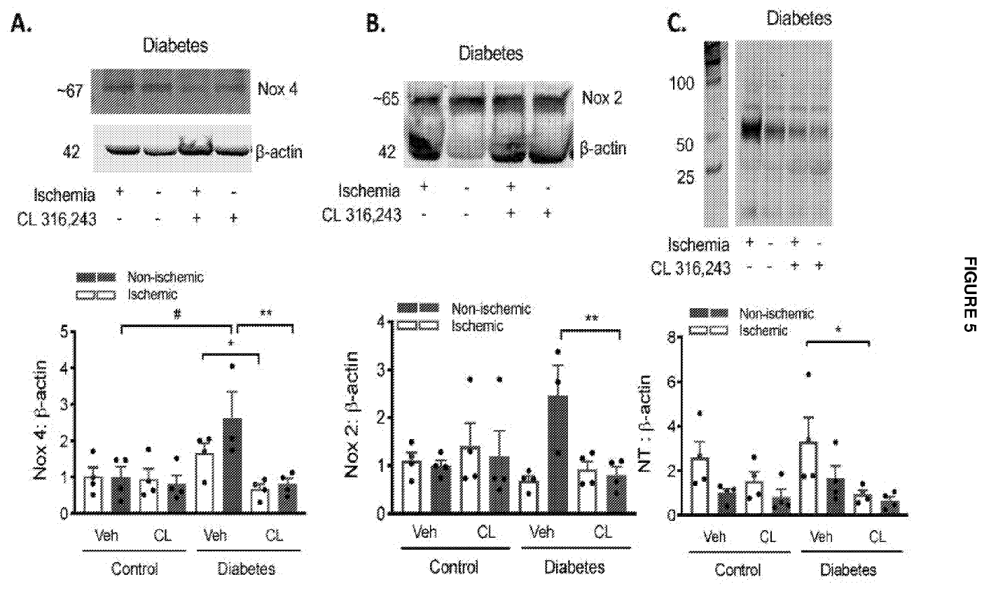

β3AR stimulation can modulate redox-NO balance. The inventors therefore examined multiple readouts important in regulating this pathway including assessment of NOX expression and levels of nitrotyrosine, a surrogate marker of reactive nitrogen species such as peroxynitrite. Compared to control, Nox 4 expression was elevated ˜2-3 fold in diabetes, in both the ischemic and non-ischemic limb (FIG. 5A). However, this diabetes-induced elevation in Nox4 expression was markedly reduced with CL treatment, back to control levels. Similar findings were observed for Nox 2 protein expression, but changes were only observed in the non-ischemic limb (FIG. 5B). Consistent with changes to the redox state in diabetes, nitrotyrosine protein levels were increased 4-fold in ischemic hind limbs of type 1 diabetes mice relative to non-ischemic limbs in control mice. β3AR agonist treatment profoundly protected against ischemia-induced nitrotyrosylation, decreasing levels by >70% in the diabetic mice (FIG. 5C). These findings indicate that β3AR agonist stimulation normalizes the dysregulated redox balance in diabetes.

### Example 6: 133AR Stimulation Abrogates eNOS Glutathionylation in Ischemic Limbs of Diabetic Mice

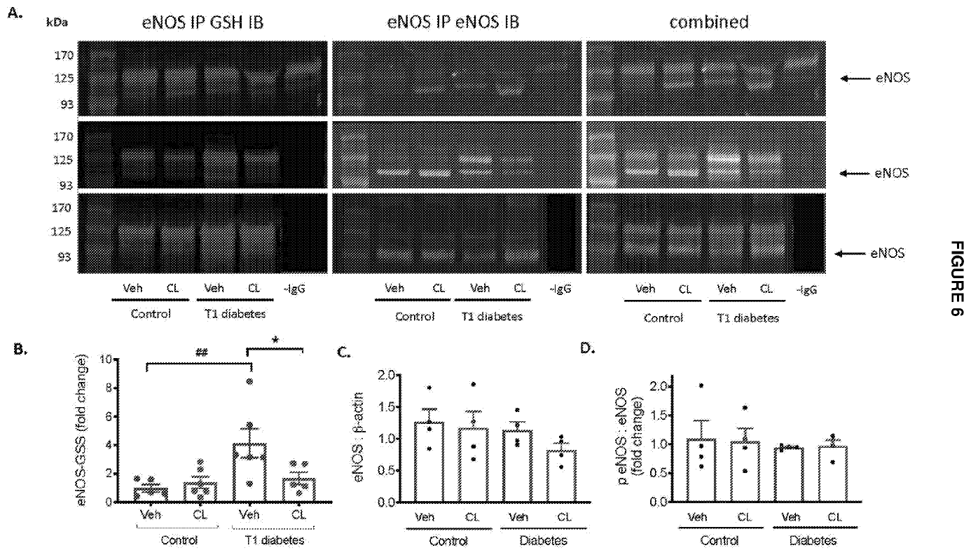

A key mechanism of eNOS uncoupling is post-translational modification involving glutathione adduct cysteine residues on the reductase domain of eNOS33. Biochemical studies performed to quantify the effect of eNOS uncoupling by this mechanism show a decrease in NO production by ˜70%, and an increase in superoxide by 5-fold 33. To investigate the possible role of eNOS glutathionylation in the ischemic limbs, and the benefits of CL 316,243, we performed eNOS immunoprecipitation and detected the oxidised glutathione and eNOS co-expression. Glutathionylation of eNOS was increased >3 fold in the ischemic limbs of type 1 diabetes mice and this was largely abolished in mice treated with the β3AR agonist (FIG. 6A). eNOS expression was unaltered (FIG. 6B). Phosphorylation of eNOS at serine 1177, which is sensitive to oxidative stress and can result in eNOS uncoupling, did not appear to be affected by either type 1 diabetes or b3AR stimulation (FIG. 6D).

### Example 7: β3AR Stimulation Also Promotes Reperfusion in a High-Fat Fed Diabetic Model

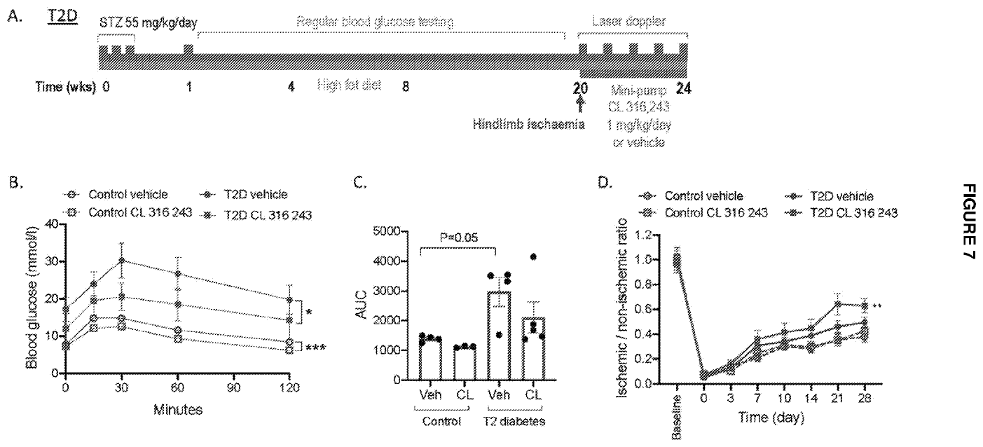

The inventors determined to investigate the effect in a model that recapitulates features of type 2 diabetes. We utilised a well-validated and characterised model of insulin resistance and type 2 diabetes. Mice were fed a high-fat diet for 20 weeks after instigation of low-dose STZ (FIG. 7A). This resulted in a hyperglycemic model that were protected from the metabolic disturbance causing substantial weight-loss seen in the type 1 diabetes model (data not shown). The body weights were similar in citrate-buffer and type 2 diabetes mice prior to hind limb ischemia, and not affected by CL 316,243 infusion after the ligation surgery (data not shown). Blood glucose levels rose rapidly and were consistently in the hyperglycemic range for the duration of the protocol. Prior to and after CL 316,243 infusion, non-fasted blood glucose levels were similar in both type 2 diabetes groups. Interestingly, when mice were fasted for glucose tolerance tests, blood glucose levels appeared lower in the CL 316,243 group, although this did not reach significant difference. Glucose tolerance was improved in type 2 diabetic mice treated with CL 316,243 (FIG. 7B). Glucose tolerance was even improved in CL 316,243 treated non-hyperglycemic, non-diabetic controls (FIG. 7C). Importantly, the protective effects of β3AR stimulation on diabetic ischemic injury were again evident, with augmented reperfusion post-ischemia in type 2 diabetes mice treated with CL 316,243 (FIG. 7D).

### Summary

There is a clear unmet need for medical treatment options targeting underlying PAD mechanisms driving both atherosclerosis as well as tissue ischemia to improve quality of life for PAD patients and reduce morbidity and mortality. Whilst surgical and percutaneous approaches to revascularization have been partially successful, this is expensive and not without risk to the patient, including the need for recurrent procedures. Therapeutic angiogenesis and improvements of microvascular function are a promising strategy. Here the inventors demonstrate for the first time that β3AR stimulation improves NO/redox balance in a pre-clinical model of PAD and this translates to significant improvement in limb perfusion in mice with vascular complications of diabetes. In addition to restoration of NO/redox balance CL 316,243 stimulated growth of new blood vessels. There may also have been contribution of vasodilation as there was evidence of systemic improvements in perfusion not specific to the ischemic limb. Whilst many preclinical studies have been challenging to translate to humans, the safe and well accepted use of the β3AR agonist, Mirabegron, for patients with overactive bladder syndrome makes the opportunity for drug repurposing and translation of our findings immediately feasible. Thus, the inventors' findings have direct relevance for the >200 million people worldwide suffering from atherosclerotic PAD, particularly those with the co-morbidity of diabetes.

The inventors' findings provide clear evidence that β3AR stimulation can promote angiogenesis in vitro, in cultured microvascular, umbilical vein and ECFCs, consistent with previous reports from studies using retinal endothelial cells. The inventors demonstrated that the pro-angiogenic effects of the β3AR agonist are due, at least in part, to improved NO bioavailability.

The inventors are the first to demonstrate the functional outcome in a model of PAD. Their demonstration of the pro-angiogenesis capacity of the β3AR agonist in relevant ECFCs from patients with cardiovascular disease provides proof-of-concept that β3AR stimulation may be effective in patient populations. These surprising findings revealed that significant angiogenesis in response to CL 316,243 did not occur in the cells from relatively healthy participants. This may indicate that β3AR stimulation is more effective in a pathological state and is supported by our animal studies showing a stronger role for β3AR stimulation in diabetic compared to healthy mice. Whilst this may be due to numerous modifications in inflammatory and oxidative signaling under disease conditions, it is likely to be at least partially dependent on the restoration of low NO bioavailability and redistribution or upregulation of β3ARs.

### Example 8: Topical Application of a Gel Comprising Mirabegron

A lipogel comprising the β3AR agonist Mirabegron was applied to freshly explanted skin for 5.5 hrs. Skin was then snap frozen in liquid nitrogen and cryosectioned at 40 μum thickness.

DESI-Mass spec was then used to scan the tissue at 30 μm resolution. Tissue image was constructed using mass signal 284 which was presented in high quantity throughout the tissue. Mirabegron was detected using mass signal 397 and overlayed onto the tissue image. The same slide used for DESI-Mass spec was subsequently stained with H&E.

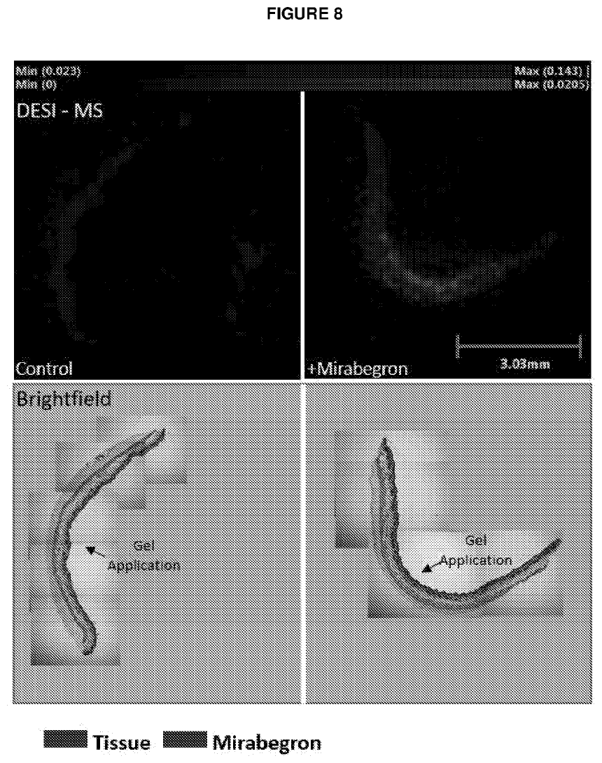

As shown in FIG. 8 Mirabegron was detected in the tissue, demonstrating successful absorption of the active agent.

### Example 9: Effect of Mirabegron on the Migration of Healthy and Diabetic HUVECs

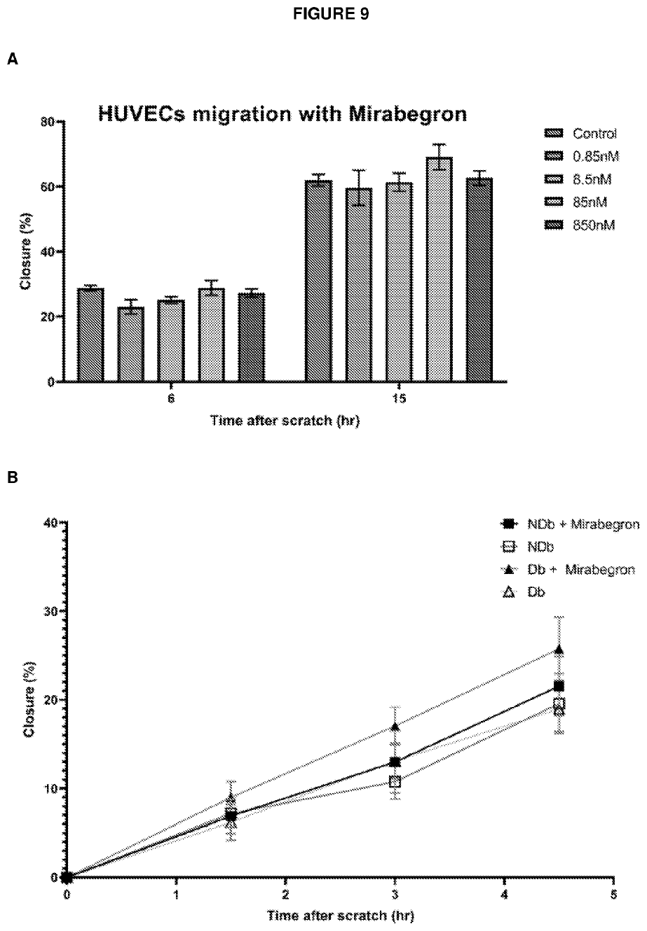

To first investigate whether the role that β3AR activation plays in endothelial cell migration was dose dependant, the inventors measured migration of HUVECs in media with multiple concentrations of Mirabegron (0.85, 8.5, 85 & 850 nM) using a scratch migration assay. No significant difference was observed between the various concentrations (FIG. 9A) which is likely due to a lack of underlying presence of oxidative stress to cause endothelial dysfunction and impaired migration.

To investigate the potential for Mirabegron to improve migration by reversing oxidative stress, diabetic and non-diabetic HMVECs were used in a scratch migration assay. 85 nM of Mirabegron was used with a control on both cell types. The results showed that the presence of Mirabegron improved the migration of both the diabetic and non-diabetic HMVECs within a 4.5-hour time frame (FIG. 9B). Diabetic HMVECs displayed a trend of increased capacity for migration when treated with Mirabegron compared to no-treatment, suggesting that Mirabegron may significantly improve migration.

### Example 10: Topical Mirabegron Lipogel Application Improves Wound Healing in a Male STZ-Diabetic Mouse Model

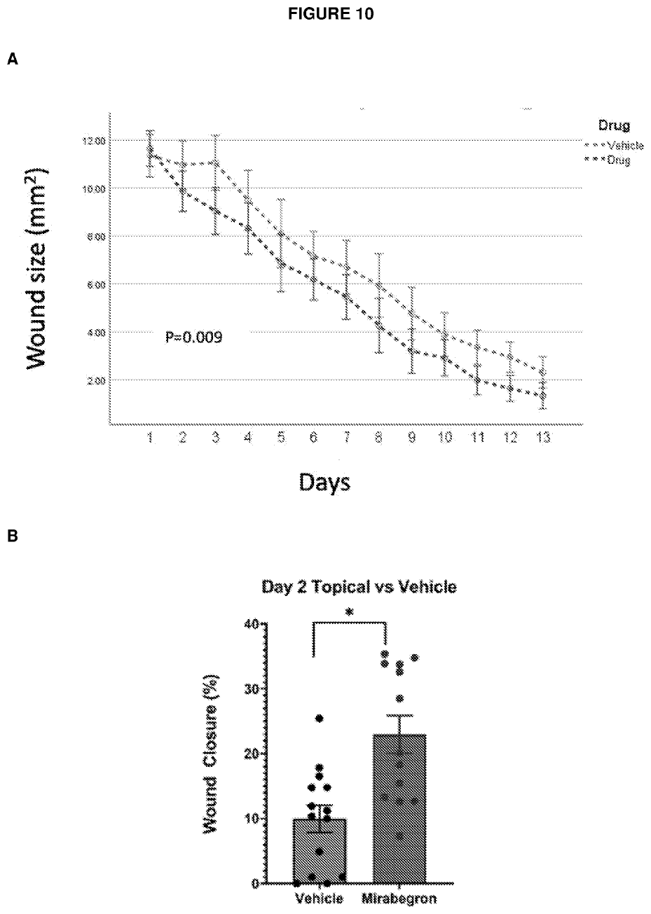

There may be considerable variability in the concentration of a systemically delivered drug compound arriving at the site of a peripheral wound due to decreased perfusion because of diabetes vasculopathy. As a result, the inventors administered Mirabegron in a topical lipogel format to better assess the effect of β3AR activation on wound healing in a diabetic mouse model. Topical application of mirabegron significantly improved wound healing as shown in FIG. 10 (p=0.009, n=12-14; 1-way ANOVA with repeated measures). Early improvements were seen, including with significance at day 2 (FIG. 7-right panel; n=14, p=0.0238) and seemed to improve wound healing from day 1 resulting in a higher percentage closure at the endpoint of day 12 when compared to control. Specifically, wounds treated with Mirabegron lipogel showed a two-fold greater rate of early closure when compared to vehicle up to day 2.

### Summary

This study found that Mirabegron lipogel topically delivered to wounds is effectively absorbed into the dermal layer where it may be able to reverse the oxidative stress causing endothelial dysfunction thus potentially restoring capacity for angiogenic wound healing. Specifically, Mirabegron seemed to improve the capacity for migration of cultured diabetic endothelial cells in vitro within a short timeframe. Furthermore, Mirabegron topically applied to mouse skin in a lipogel emulsion was absorbed into the dermal layers, establishing an opportunity for a novel targeted delivery mechanism for Mirabegron. Combining these ideas, the inventors were able to show that topical application of Mirabegron lipogel significantly improved early wound healing in a male STZ-diabetes mouse model using a splinted wound model.

Through the in vitro scratch migration assays, the inventors found that Mirabegron (0.85 nM-850 nM) had no impact on the migration of cultured HUVECs over a 15-hour period. Contrastingly, Mirabegron (85 nM) seemed to improve the migration of cultured diabetic HMVECs over a shorter 4.5-hour period while having no effect on migration of non-diabetic HMVECs. Without wishing to be bound by theory, the inventors believe that the pro-migration effects of β3 activation are partially due to the increase in NO bioavailability resulting from the direct coupling of β3AR with eNOS; this agrees with the observed trend suggesting Mirabegron's effect on diabetic HMVECs, in which hyperglycemia-driven oxidative stress decreases the availability of NO, but not on non-diabetic HMVECs exhibiting homeostatic NO levels.

Proof-of-concept DESI MS scanning of mouse tissue incubated with topically applied Mirabegron in a lipogel emulsion demonstrated that Mirabegron could be absorbed into the dermal layers in a short period. The recent shift in understanding of DFUs as a primarily cardiovascular pathology as opposed to neuropathic has driven a new generation of treatments reflecting this paradigm shift. Similarly, the decreased perfusion of wound tissue contributing to difficulty in systemic treatment of DFUs has led to testing of more effective targeted delivery approaches, namely topical delivery in preclinical wound healing models. To the inventors' knowledge, this study is the first to uniquely marry these existing concepts with the recent discovery of β3AR's role in endothelial function and test the effect of topical application of a β3AR agonist on a pre-clinical wound healing model.

This study found a significant improvement in closure of a splinted wound with daily topical application of Mirabegron lipogel in a diabetic mouse model at day 2. This finding indicates that Mirabegron at least partially plays a role in the early stages of diabetic wound healing.

It will be understood that the invention disclosed and defined in this specification extends to all alternative combinations of two or more of the individual features mentioned or evident from the text or drawings. All of these different combinations constitute various alternative aspects of the invention.

1. A method for promoting wound healing in a subject, the method comprising the step of administering a therapeutically effective amount of a β3-Adrenergic Receptor (βAR) agonist to a subject in need thereof, thereby promoting wound healing in the subject.

2. The promotion of wound healing may be for the prevention, pre-emptive therapy and/or treatment of a dermal or cutaneous wound, or other wound of the mucous membranes or connective tissues of the subject.

3. A method for the treatment of a dermal or cutaneous wound, the method comprising the step of administering a therapeutically effective amount of a β3AR agonist to a subject in need thereof, thereby treating the dermal or cutaneous wound.

4. A method for promoting revascularisation and blood supply to a wound, the method comprising administering to a subject in need thereof, a therapeutically effective amount of a β3AR agonist.

5. A method of accelerating the rate of wound healing, or decreasing the time to completion of wound healing or wound closure, the method comprising the step of administering a therapeutically effective amount of a β3-Adrenergic Receptor (β3AR) agonist to a subject in need thereof, thereby accelerating the rate of wound healing, or decreasing the time to completion of wound healing or wound closure.

6. The method of any one of claims 1 to 5, wherein the β3AR agonist is administered orally, intravenously, intraarterially, intradermally, subcutaneously or topically.

7. The method of any one of claims 1 to 5, wherein the method comprises contacting the wound with the β3AR agonist.

8. The method of claim 7, wherein the β3AR agonist is administered in the form of a gel, lotion, cream, impregnated sponge, ointment or spray or via intradermal or subcutaneous injection.

9. A method for promoting the healing of a dermal or cutaneous wound, the method comprising the step of contacting a dermal or cutaneous wound with a therapeutically effective amount of a β3AR agonist, thereby promoting the healing of the dermal or cutaneous wound.

10. The method of claim 9, wherein the method comprises decreasing the wound area or volume of the dermal or cutaneous wound.

11. The method of claim 9, wherein the method comprises accelerating the rate of wound healing, or decreasing the time to completion of wound healing or wound closure.

12. The method of any one of claims 1 to 11, wherein the wound is in a subject who has or is at risk of impaired wound healing.

13. The method of claim 12, wherein the subject has, or is considered at risk of a vascular disease or condition, such as: peripheral arterial disease (PAD), scleroderma and/or atherosclerosis.

14. The method of claim 12, wherein the subject has type I or type II diabetes.

15. The method of any one of the preceding claims, wherein the wound is an acute wound.

16. The method of any one of claims 1 to 15, wherein the wound arises from pressure, laceration, burn, incision, maceration, crushing, puncture abrasion or like injury.

17. The method of any one of claims 1 to 14, wherein the wound is a chronic wound.

18. The method of claim 17, wherein the wound is associated with a vascular condition characterised by decreased blood circulation or blood flow.

19. The method of claim 18, wherein the wound is a venous leg ulcer, a venous foot ulcer, an arterial leg ulcer, an arterial foot ulcer or a decubitus ulcer (also known as a pressure ulcer, bed sore or pressure sore).

20. The method of claim 19, wherein wound is associated with diabetes mellitus.

21. The method of claim 20, wherein the wound is a diabetic foot ulcer.

22. Use of a β3AR agonist in the manufacture of a medicament for:
promoting wound healing;
the treatment of a dermal or cutaneous wound;
inducing or promoting angiogenesis and blood flow to a wound;
decreasing the wound area or volume of a dermal or cutaneous wound;
accelerating the rate of wound healing, or decreasing the time to completion of wound healing or wound closure;
inducing or promoting or initiating a wound repair mechanism in a dermal or cutaneous wound; and/or
treating or managing a diabetic ulcer, preferably a diabetic foot ulcer.

23. A β3AR agonist for use in:
promoting wound healing;
the treatment of a dermal or cutaneous wound;
inducing or promoting angiogenesis and blood flow to a wound;
decreasing the wound area or volume of a dermal or cutaneous wound;
accelerating the rate of wound healing, or decreasing the time to completion of wound healing or wound closure;
inducing or promoting or initiating a wound repair mechanism in a dermal or cutaneous wound; and/or
treating or managing a diabetic ulcer, preferably a diabetic foot ulcer.

24. A pharmaceutical composition comprising a β3AR agonist for use in:
promoting wound healing;
the treatment of a dermal or cutaneous wound;
inducing or promoting angiogenesis and blood flow to a wound;
decreasing the wound area or volume of a dermal or cutaneous wound;
accelerating the rate of wound healing, or decreasing the time to completion of wound healing or wound closure;
inducing or promoting or initiating a wound repair mechanism in a dermal or cutaneous wound; and/or
treating or managing a diabetic ulcer, preferably a diabetic foot ulcer.

25. The method of any one of claims 1 to 21, the use of claim 22, the β3AR agonist for the use of claim 23, or the composition of claim 24, wherein the β3AR agonist is selected from the group consisting of: amibegron (SR-58611 A, Sanofi); BRL-37344; CL-316,243; L-742,791; L-796,568; LY-368,842, Mirabegron (YM-178), Nebivolo, Ro40-2148, Solabegron (GW-427,353, GSK); Vibegron (MK-4618, Kyorin Pharmaceutical Co., Ltd, and Kissei Pharmaceuticals Co Ltd); and Ritobegron (KUC-7483; Kissei Pharmaceuticals Co Ltd).

26. The method, use, β3AR agonist, or the composition of claim 25, wherein the β3AR agonist is Mirabegron, or a pharmaceutically acceptable salt thereof.

27. The method, use, β3AR agonist, or the composition of claim 25, wherein the β3AR agonist is CL316,243, or a pharmaceutically acceptable salt thereof
